(19) United States
(12) Patent Application Publication
(10) Pub. No.: US 2021/0322662 A1
(43) Pub. Date: Oct. 21, 2021

(54) A Control And/Or Closed-Loop Control Device For Removing Fluid from A Blood Filter

(71) Applicant: Fresenius Medical Care Deutschland GmbH, Bad Homburg (DE)

(72) Inventor: Martin Thys, Grettstadt (DE)

(21) Appl. No.: 17/258,856

(22) PCT Filed: Jul. 9, 2019
(86) PCT No.: PCT/EP2019/068403

§ 371 Date: Jan. 8, 2021

(30) Foreign Application Priority Data
Jul. 11, 2018 (DE) 10 2018 116 806.3

(51) Int. Cl. A61M 1/36; A61M 1/34; A61M 39/28

(52) U.S. Cl. CPC A61M 1/3646; A61M 1/3424; A61M 2205/3341; A61M 39/28; A61M 1/3649

(57) ABSTRACT

The present disclosure relates to a control and/or closed-loop control device for executing a method for removing fluid from an extracorporeal blood circuit, in particular from a blood filter and/or from a venous air separation chamber, used for the blood treatment of a patient, wherein said controlling or closed-loop controlling is carried out after the completion of the blood treatment or the blood treatment session. It further relates to a medical treatment apparatus having a control and/or closed-loop control device with which the method according to the present disclosure is executable, a digital storage medium, a computer program product and a computer program.

### CROSS-REFERENCE TO RELATED APPLICATIONS

The present application is the national stage entry of International Patent Application No. PCT/EP2019/068403, filed on Jul. 9, 2019, and claims priority to Application No. DE 10 2018 116 806.3, filed in the Federal Republic of Germany on Jul. 11, 2018, the disclosures of which are expressly incorporated herein in their entirety by reference thereto.

### FIELD OF THE INVENTION

The present disclosure relates to a control and/or closed-loop control device configured to effect a method for removing fluid from a blood filter for the extracorporeal blood treatment of a patient after the completion of the blood treatment session. It further relates to a medical treatment apparatus. In addition, the present disclosure relates to a digital storage medium, a computer program product, as well as a computer program.

### BACKGROUND

Blood filters and extracorporeal blood circuits are usually disposables and are disposed of after their use. Disposing is cost-intensive and has to be paid by weight of waste. For this reason, and also in order to reduce the risk of contamination, these disposables are thus emptied from fluid and in particular from blood prior to their disposal.

### SUMMARY

It is an advantage of the present disclosure to specify a further control and/or closed-loop control device for effecting, after the completion of a blood treatment or a blood treatment session, a method for removing fluid from a blood filter or from a blood chamber thereof and if necessary from an extracorporeal blood circuit connected thereto. Additionally, suitable apparatuses shall be specified.

Advantages according to the present disclosure are achieved by a control and/or closed-loop control device disclosed herein. Advantages are further accomplished by the medical treatment apparatus, the digital storage medium, the computer program product, as well as the computer program that are all disclosed herein.

Thus, according to the present disclosure, a control and/or closed-loop control device which after the completion of the blood treatment session, effects a removing of fluid, in particular blood, or a part thereof from an extracorporeal blood circuit used for the blood treatment of a patient.

The extracorporeal blood circuit from which fluid is removed by controlling or regulating using the control and/or closed-loop control device according to the present disclosure comprises a blood filter or is connected thereto in fluid communication. The blood filter comprises a blood chamber and a dialysate chamber, between which a mostly semipermeable membrane is arranged. For the blood treatment, the blood chamber is connected to an arterial blood line (i.e. to the blood withdrawal line; these terms are herein interchangeable) leading to the blood chamber and to a venous blood line (i.e. to a blood return line; these terms are herein interchangeable) leading away from the blood chamber. The dialysate chamber is connected to a dialysis liquid inlet line leading to it and to a dialysate outlet line leading away from it. Furthermore, the extracorporeal blood circuit comprises a valve which serves for either establishing or interrupting a fluid connection between the interior of the extracorporeal blood circuit and its exterior or the atmosphere.

The medical treatment apparatus on which the control and/or closed-loop control device according to the present disclosure acts comprises at least one blood pump arranged at or in the extracorporeal blood circuit for conveying blood within the line interior of the extracorporeal blood circuit. In addition, it comprises at least one conveying device which serves for introducing substituate or dialysis liquid into the arterial blood withdrawal line or into the venous blood return line. The medical treatment apparatus further comprises a venous patient hose clamp and/or an actuation device designated thereto, with which a flow through a section of the venous blood return line may be interrupted. Furthermore, it comprises an actuation device configured to actuate the above-described valve of the extracorporeal blood circuit, in particular to open or to close it (by which a flow may be interrupted). At least one pressure sensor, arranged for measuring the pressure prevailing in the arterial blood withdrawal line or in the venous blood return line, is also comprised by the medical treatment apparatus.

The method which is effected by the control and/or closed-loop control device according to the present disclosure encompasses introducing substituate (alternatively: substitution liquid) or dialysis liquid for displacing the fluid, in particular the blood, from the extracorporeal blood circuit, in particular from the blood filter and from its blood chamber. The method encompasses the following steps:
a) interrupting a connection between the atmosphere or the exterior on the one hand and an interior of the extracorporeal blood circuit on the other hand by closing the valve discussed above;
b) closing the venous patient hose clamp;
c) introducing substituate or dialysis liquid into the extracorporeal blood circuit, in particular into the arterial blood withdrawal line or into the venous blood return line, and/or establishing an increased pressure by the pump used for introducing;
d) optionally: measuring the pressure prevailing in the arterial blood withdrawal line and/or in the venous blood return line, e.g. by the aforementioned pressure sensor;
e) establishing the fluid connection between the atmosphere or the exterior and the interior of the blood circuit by opening the a.m. valve, when or if the measured pressure has reached or exceeded a predetermined, first value, or when or if a predetermined first time period has elapsed since the beginning of the introduction of substituate or since the beginning of establishing pressure with a closed venous patient hose clamp (so-called “time regulation”);
f) interrupting the fluid connection between the atmosphere/the exterior on one hand and an interior of the blood circuit on the other hand by closing the valve and opening the patient hose clamp, both when, i.e. for example after or as soon as the measured pressure has dropped to a predetermined second value or when, i.e. after, a predetermined second time period has elapsed since the valve was opened in step e).

The medical treatment apparatus according to the present disclosure (hereafter also in short: treatment apparatus) comprises at least one extracorporeal blood circuit with a line interior. It is further equipped with at least one blood pump for conveying blood within the line interior of the extracorporeal blood circuit, the blood pump being arranged at or in the extracorporeal blood circuit. In addition, it comprises at least one conveying device, for instance a substituate or dialysis liquid pump, provided for introducing substituate or dialysis liquid into the arterial blood withdrawal line. Moreover, it comprises at least one control or closed-loop control device according to the present disclosure.

A storage device according to the present invention, in particular a digital one, in particular a non-volatile one (denoted here also as carrier), in particular in the form of a disk, RAM, ROM, CD, hard disk, DVD, USB stick, flash card, SD card, or EPROM, in particular with electronically or optically readable control signals may be configured such that to configure a control and/or closed-loop control device to a control and/or closed-loop control device according to the present disclosure with which the method described supra may be effected.

In this, all, several or some of this machine-inducible method steps may be prompted.

A computer program product according to the disclosure comprises a volatile program code or a program code saved on a machine-readable carrier which configures a control and/or closed-loop control device to a control and/or closed-loop control device according to the present disclosure with which the method described supra may be effected.

In this, all, several or some of this machine-inducible method steps may be prompted.

The term “machine-readable carrier”, as used herein, denotes in certain embodiments according to the present disclosure a carrier which contains data or information which is interpretable by software and/or hardware. The carrier may be a data carrier such as a disk, a CD, DVD, a USB stick, a flashcard, an SD card an EPROM and the like.

A computer program according to the present disclosure comprises a program code for configuring a control and/or closed-loop control device to a control and/or closed-loop control device according to the present disclosure with which the method described supra may be effected.

In this, all, several or some of this machine-inducible method steps may be prompted.

According to the present disclosure a computer program product can be understood as, for example, a computer program which is stored on a data carrier, an embedded system as a comprehensive system with a computer program (e.g. an electronic device with a computer program), a network of computer-implemented computer programs (e.g. a client-server system, a cloud computing system, etc.), or a computer on which a computer program is loaded, executed, saved or developed.

A computer program according to the present disclosure can be understood as, for example, a physical software product, which is ready for distribution and has a computer program.

In all of the following statements, the use of the expression “may be” or “may have” and so on, is to be understood synonymously with “preferably is” or “preferably has,” and so on respectively, and is intended to illustrate an embodiment according to the present disclosure.

Whenever numerical words are mentioned herein, the person skilled in the art shall recognize or understand them as indications of numerical lower limits. Hence, unless it leads to a contradiction evident for the person skilled in the art, the person skilled in the art shall comprehend for example “one” as encompassing “at least one”. This understanding is also equally encompassed by the present disclosure as the interpretation that a numerical word, for example, “one” may alternatively mean “exactly one”, wherever this is evidently technically possible in the view of the person skilled in the art. Both of these understandings are encompassed by the present disclosure and apply herein to all used numerical words.

Whenever the terms “programmed” or “configured” are mentioned herein, it is thus disclosed that these terms are interchangeable.

Whenever a suitability or a method step is mentioned herein, the present disclosure encompasses a corresponding programming or configuring of a suitable apparatus or a section thereof as well as apparatuses programmed in such a manner.

Some of the advantageous developments of the present disclosure are in the subject-matter of the dependent claims and embodiments.

Whenever an embodiment is mentioned herein, it is then an exemplary embodiment according to the present disclosure.

Whenever reference is made to time, duration or the like, encompassed therewith is the presence of corresponding, suitable time measuring devices.

Whenever the completion of a blood treatment or of a blood treatment session is mentioned herein, this may refer to the point of time at which there is no longer patient blood delivered to the blood treatment apparatus, e.g. to the blood filter, or at which no blood is withdrawn from the patient.

Whenever reference is made to establishing an increased pressure by the pump used for introducing, this may be understood to be an increase of the pressure that prevailed until the beginning of the increase. Establishing an increased pressure may thus be an increase of pressure.

Hence, establishing a pressure or establishing an increased pressure may preferably not be understood as a decreasing or a lowering of pressure and also not as maintaining a constant pressure.

Embodiments according to the present disclosure may comprise one or several of the features mentioned supra and/or in the following in any combination which is technically possible.

In some embodiments of the control and/or closed-loop control device, the first predetermined value for the pressure to open the valve of the medical treatment apparatus is e.g. between 1 and 2 bar.

In other embodiments of the control and/or closed-loop control device, the valve of the medical treatment apparatus is opened after a predetermined first time period being preferably between 2 seconds (sec) and 5 seconds (sec).

In some embodiments of the control and/or closed-loop control device, the pressure by which the valve of the medical treatment apparatus is closed again drops to or below a second predetermined value, e.g. between 0 and 0.5 bar.

In other embodiments of the control and/or closed-loop control device, the valve of the medical treatment apparatus is closed after a predetermined second time period being preferably between 1 sec and 3 sec.

When introducing substituate or dialysis liquid into the extracorporeal blood circuit and/or establishing an increased pressure, energy is stored due to the compliance of the hose system that allows storing energy by elastic deformation hose sections. The compliance acts therefore as an energy saver. Opening the aforementioned valve will discharge and release the energy saved due to the compliance. This may advantageously contribute to the sudden release of fluid or to the increased flow velocity when the valve is opened.

In several embodiments, when the method is executed by the control and/or closed-loop control device according to the present disclosure, a section, e.g. a free end or an end on the patient side, of the arterial blood withdrawal line is brought into fluid communication with a further section of the venous blood return line. The further section may be provided on a blood cassette. It may be equipped with a connection section and prepared to be connected to e.g. an optional connector (e.g. Luer connector, bayonet connector) of the arterial blood withdrawal line.

The connection serves for emptying the arterial section of the extracorporeal blood circuit into the venous blood return line, e.g. upstream of the addition site for substituate (predilution).

An existing or targeted pressure may be maintained in the arterial line optionally also by other measures, e.g. by an occluding pump, e.g. a blood pump, or by closing an arterial patient hose clamp, and thus preventing a decrease of pressure through or over or on the arterial side of the blood circuit.

In some embodiments, the extracorporeal blood circuit of the treatment apparatus, on which the control and/or closed-loop control device acts, comprises a venous air separation chamber or blood chamber (which is not part of the blood filter). In some of these embodiments, the a.m. method begins with the steps a) to f), only after e.g. suitable sensors, like optical sensors, confirm that the venous air separation chamber is not completely filled with liquid, in particular blood or not filled with liquid beyond a predetermined level.

In several embodiments, the extracorporeal blood circuit comprises both a venous air separation chamber and a single-needle chamber. The valve for establishing a fluid connection is hereby optionally arranged between the single-needle chamber and the venous air separation chamber. These two chambers and the valve may in turn be arranged on a blood cassette or be part thereof.

In some embodiments, an arbitrary compensation tank or container is arranged instead of the single-needle chamber or is fluidly opened or connected by opening the valve.

In several embodiments, after or subsequently to step f) the little or small amount of liquid is actively discharged, e.g. by a single-needle compressor already exiting in some dialysis machines like the 6008 by Fresenius, Germany.

In some embodiments, the steps a) to f) of the method, which is effected by the control and/or closed-loop control device according to the present disclosure, are executed successively several times.

For a high efficiency, the steps b) to e) may be repeated during the period or duration of blood return, typically within three to four minutes, at optional intervals, several times, preferably five to fifteen times or more.

In several embodiments, at least one section of the extracorporeal blood circuit of the medical treatment apparatus according to the present disclosure is part of a blood cassette.

Such a cassette is exemplarily described in the WO 2010/121819 A1 of the applicant of the present application. Its relevant content is hereby thoroughly incorporated by reference as a subject-matter also of the present application.

Such a blood cassette may comprise at least a housing body, preferably made of a hard plastic, having at least one, two, or more chamber(s) for receiving medical fluids integrated into the housing body, at least one channel integrated into the housing body for receiving and/or conducting a medical fluid, and at least one valve device completely or partially integrated in the housing body for controlling or regulating a fluid flowing through the blood cassette.

Such a blood cassette may comprise at least one single-needle chamber for receiving blood downstream of the blood filter. Such a single-needle chamber may encompass or have at least one single-needle valve, or be in fluid connection thereto, which controls or regulates the inflow and/or outflow of blood in the single-needle chamber. Examples of a single-needle chamber and a single-needle valve are exemplarily described in WO 2010/121819 A1, whose content is entirely incorporated by reference as a subject-matter of the present disclosure as well.

Such a blood cassette may comprise at or on at least one of its surfaces a covering device or cover, e.g. a film, which is preferably part of at least one integrated valve device.

With such a blood cassette, the cover device, e.g. as a film, may be at least in one section connected with the housing body in a force-fit and/or form-fit and/or material-bonded manner.

With such a blood cassette, the cover device may be connected to the housing body by at least one closed or circumferential weld seam.

The single needle chamber may be an active or non-active part in the blood circuit by correspondingly switching an optional single needle valve, which separates the single needle chamber preferably from further structures, in particular from all further blood conducting structures. When the single needle chamber is accessible—by opening the single needle valve—for blood or another fluid which flows out of the venous section downstream of the blood filter and when at the same time a flow-through of blood or of fluid along the single-needle chamber is prevented, exemplarily by closing the venous patient hose clamp, then blood may first be buffered or stored in the single-needle chamber before it leaves the single-needle chamber towards the venous patient hose clamp after the venous patient hose clamp has been opened.

The utilized blood filter is in some embodiments according to the present disclosure a hemodialyzer.

In some exemplary embodiments according to the present disclosure, the method encompasses displacing the fluid from the blood chamber by introducing a substituate or dialysis fluid into the arterial blood withdrawal line with a flow rate of the substituate or the dialysis liquid which, at least in phases, exceeds 150 ml/min, 200 ml/min, 250 ml/min, 300 ml/min, 350 ml/min, 400 ml/min, 450 ml/min, 500 ml/min, 550 ml/min and/or is equal to or less than 600 ml/min.

The sequence or order of the steps a) to f) may optionally be fixed or binding, in particular step f) preferably follows only step e).

In several embodiments, the sequence of the steps a) to f) takes preferably a maximum of 5 to 10 seconds. Switching times for valves and clamps may be included herein.

In some embodiments, during the sequence of step a) to f), there is no increase of the conveying velocity at which substituate or dialysis liquid is conveyed by the respective conveying device. However, such an increase, which is omitted in these embodiments, could be present if it were measurable using flow meters upstream of a substituate addition site. It could also be present if a setting or adjustment of the conveying rate or velocity would have been changed by a control device of the conveying device. The set or targeted conveying rate or velocity thus remains unchanged and/or constant.

Preferably, it is ensured that the venous air separation chamber is not completely filled with blood prior to the start of the blood return, during which this method is being executed.

In some embodiments, there is not active conveying into the exterior, e.g. into the compensation tank, during a sequence of the steps a) to f). In these embodiments, filling the compensation tank with liquid happens preferably only or substantially only due to the pressure relief when opening the valve.

Preferably, after starting the blood return to the patient, e.g. by a pump, liquid is conveyed towards the dialyzer, preferably without having to stop or interrupt the performance of the pump for introducing substituate or dialysis liquid during steps a) to f).

In some exemplary embodiments according to the present disclosure, the connection between the arterial blood withdrawal line and the venous blood return line is established, in particular exclusively for the purpose of emptying the blood filter.

A “blood treatment session” may be, for example, a treatment unit by hemodialysis, hemofiltration, hemodiafiltration and/or a cell separation method and it is directed to the treatment and/or purification of blood. For performing such a blood treatment, a suitable blood treatment apparatus is used.

Conveying devices for introducing substituate or dialysis liquid, as mentioned herein, include membrane pumps, hose pumps, roller pumps and so on. The blood pump, a substituate pump and/or a dialysis fluid pump may be embodied, e.g., as a hose pump or a roller pump. However, also a different type of pump may be used, e.g. a membrane pump, particularly a high-precision metering membrane pump.

A conveying device for dialysis fluid or substituate may be a “second” conveying device, i.e. a conveying device which is different from the blood pump. The blood pump may, however, also be designed such that it fulfils both the function which is typical for a blood pump and the function of introducing substituate into the line interior and/or conveying line content. Whenever a conveying device for introducing substituate or dialysis liquid is mentioned hereafter, just for the purpose of better legibility, this relates to the blood pump or a conveying device different therefrom. Both versions are equally encompassed by the present disclosure.

“Introducing substituate or dialysis liquid into the line interior of the extracorporeal blood circuit” takes place, as described above, in some particular embodiments according to the present disclosure by operating the blood pump and/or the conveying device.

The blood pump may convey substituate by withdrawing it from a supply line from a container for the substituate, wherein the supply line opens into the extracorporeal blood circuit upstream of the suction side of the blood pump. For this, for example an opening or mouth with a hose clamp which is provided in the arterial branch of the extracorporeal blood circuit may be provided.

If the blood pump is intended or provided to introduce and convey both blood and substituate in the extracorporeal blood circuit, the method according to the present disclosure may be executed with only one pump. Even though such a further preferred embodiment is encompassed by the present disclosure, in the following, embodiments are described in which a blood pump and a second conveying device are used. The following description shall simplify the understanding of the principles and functions of the individual components on which the present disclosure is based. In this, the substituate or the dialysis liquid may be conveyed by the blood pump and/or the conveying device, for instance the substituate pump. They may be used individually or collectively for conveying and/or establishing pressure as described herein.

A “substituate” may be for example any commonly known substituate or dialysis liquid which is used during a blood treatment such as, e.g., a hemodiafiltration, preferably a solution or isotonic saline solution for example a 0.9% NaCl solution which has already been used during the blood treatment session and which thus was already introduced or is introducible into the extracorporeal blood circuit via a fluid connection.

The “section of the extracorporeal blood circuit” may be an arterial and/or venous section of the extracorporeal blood circuit. The “arterial section” relates to a section of the extracorporeal blood circuit through which blood flows from the vascular system of the patient in the direction towards the blood treatment device or towards the blood filter. The “venous section” relates to the section of the extracorporeal blood circuit through which blood flows from the blood treatment device or from the blood filter back to the vascular system of the patient.

“An addition site for the extracorporeal blood circuit for substituate into the line interior of the extracorporeal blood circuit” may be arranged in the arterial and/or the venous section of the extracorporeal blood circuit. The “addition site” is preferably arranged in a section of the extracorporeal blood circuit which is flown through or perfused upstream of the blood treatment device which may for example be the blood filter.

Suitable examples for an addition site include an opening/closing valve, a stop cock, a connectable branch line of a branched section of the extracorporeal blood circuit and so on.

“Returning blood into the vascular system of the patient” may take place if or when an end of the extracorporeal blood circuit, such as for example the end of the venous section, e.g. the venous connection needle, is connected with the vascular system of the patient. This connection may be maintained or re-established after the end of the blood treatment session.

Due to the fact that the method as described above is executable with the treatment apparatus according to the present disclosure, reference is made to the respective embodiments as described above in order to avoid repetition.

A further development of the treatment apparatus according to the present disclosure includes arranging at least one detection device for detecting at least one change of the content of the line interior of the extracorporeal blood circuit or of one property of the content in a section of the extracorporeal blood circuit. A property of the content may be a composition, a physical, a chemical or biological value, for example a transparency, a pH value and much more suchlike. A suchlike detection device may correspond to the one described above, thus reference is made to its above description in order to avoid repetition.

A treatment apparatus according to the present disclosure may, without being limited hereto, be suitable and/or configured to perform a hemodialysis, a hemofiltration, a hemodiafiltration and separation methods.

One or several of the herein mentioned advantages may be achieved by some embodiments according to the present disclosure.

Thus, the present disclosure may contribute in certain embodiments according to the present disclosure to an improvement of the effectivity of the blood return.

An advantage which can be achieved in some particular embodiments according to the present disclosure is that a contamination risk during further handling or disposing of the blood filter and/or of the extracorporeal blood circuit may be avoided or diminished, because after the method according to the present disclosure has been completed, there isn't any blood remaining in the blood filter anymore or its amount has been markedly reduced.

Emptying at least the blood filter, particularly from blood, may in certain embodiments according to the present disclosure take place without substantial interaction or without any interaction by the user, e.g., the doctor, at least until the blood filter is removed from the treatment apparatus. A side effect is that the error rate is low. In addition, the user has time for other activities during emptying. This contributes to easing the workload and saving time altogether.

Blood components adhering to the inner wall of the membrane capillaries are better detached due to the increased flow velocity and thus the increased input of kinetic energy and the associated friction.

The present disclosure may further advantageously be used for safe removal of blood from the extracorporeal blood circuit for a treatment apparatus for the extracorporeal blood treatment of a patient after the completion of a blood treatment session: Since substituate is present in the line interior of the extracorporeal blood circuit after the completion of the blood treatment session, the blood present in the line interior of the extracorporeal blood circuit can be removed from the extracorporeal blood circuit. This may take place without the risk of introducing air into the body of the patient.

Besides, it may also be possible to minimize foaming, for example at the outlet of the blood filter, by using substituate there, i.e. in particular a liquid, and not only a gas (e.g. air) for displacing blood or emptying.

Since the method which is effected by the control and/or closed-loop control device according to the present disclosure may be executed directly after a blood treatment session has been completed, it is simply and easily executable and does not require any technically complex, time- and/or cost-intensive steps.

The method which is effected by the control and/or closed-loop control device according to the present disclosure may advantageously be executed with the substituate or dialysis fluid which is used or present anyway during a blood treatment, such as for example an isotonic saline solution, e.g., a 0.9% NaCl solution. This in turn advantageously contributes to saving costs and time.

Furthermore, the method which is effected by the control and/or closed-loop control device according to the present disclosure may enable a removal of blood from the arterial section of the extracorporeal blood circuit and in particular from the arterial blood withdrawal line and the return of the blood into the vascular system of the patient. The step of retrogradely pushing out the blood present in the arterial blood withdrawal line with the aid of, e.g., a syringe which is filled with saline solution may thus advantageously be avoided.

The method which is effected by the control and/or closed-loop control device according to the present disclosure may thus offer the advantage of basically completely regaining of blood present in the line interior of an extracorporeal blood circuit after it has been used for a blood treatment for the patient.

The resulting flow tips may advantageously contribute to detaching blood from the surface of the extracorporeal blood circuit. Due to intermittently increased pressure, blood residuals are detached from structures like the blood filter. These blood residuals may thus also be reinfused to the patient.

In addition, by automatically effecting the flow tips, the need for manual intervention by the user may be omitted and, consequently, the efficiency of the method may be increased or improved.

According to the present disclosure, there are no flow tips that continue beyond the extracorporeal blood circuit up to or into the vascular system of the patient. By the controlled controlling of the course this is advantageously avoided and the vascular system of the patient is protected.

Advantageously, by intentionally opening the valve, e.g. between venous air separation chamber and compensation tank, and by the associated escape of pressure, an undesired passing on of pressure towards the vascular system which might be harmful, is automatically counteracted.

Since according to the present disclosure only a very small amount of liquid reaches or enters a compensation tank, there is no need for a subsequent active discharging of this liquid from the compensation tank. This allows to omit a pump or other conveying devices for this purpose.

### BRIEF DESCRIPTION OF THE FIGURES

In the following, the method according to the present disclosure is described based on preferred embodiments thereof with reference to the accompanied drawing. In the drawing the following applies:

FIG. 1 shows an exemplary embodiment of schematically simplified sections of a medical treatment apparatus according to the present disclosure with a blood cassette for executing the method controlled and/or closed-loop controlled by a control and/or closed-loop control device according to the present disclosure.

### DETAILED DESCRIPTION

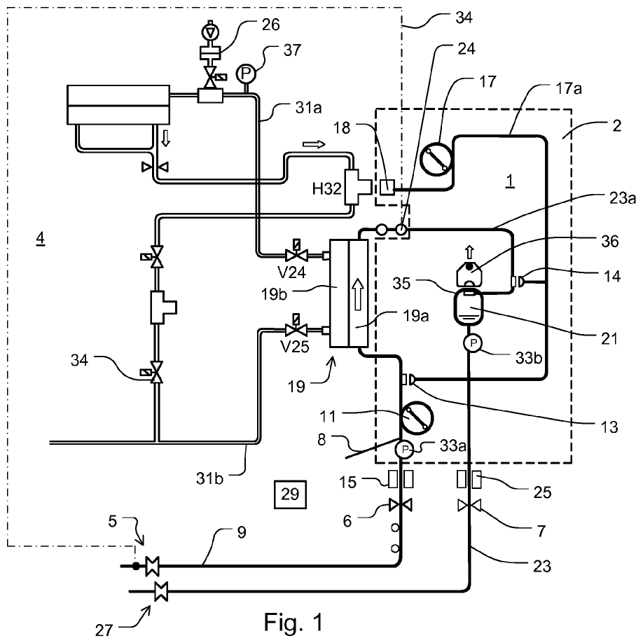

FIG. 1 shows an extracorporeal blood circuit 1, which would be connected for a treatment to the vascular system of the patient (not shown) via double-needle access or via single-needle access using e.g. an additional Y-connector. The blood circuit 1 is provided optionally in sections thereof in or on a blood cassette 2. This blood cassette 2 is designed to be used also in other treatment types, e.g. a single-needle treatment.

Pumps, actuators, and/or valves in the area of the blood circuit 1 are connected to a treatment apparatus 4 according to the present disclosure or to a control and/or closed-loop control device 29 according to the present disclosure.

The blood circuit 1 comprises an arterial patient hose clamp 6 and an arterial connection needle 5 (as an example for an access device) of an arterial section or of an arterial patient line or blood withdrawal line 9. The blood circuit 1 further comprises a venous patient hose clamp 7 and a venous connection needle 27 (as an example for a further or second access device) of a venous section or of a venous patient line or blood return line 23.

A blood pump 11 is provided in the arterial section 9, a substituate pump 17 is connected to a substituate line 17a. The substituate line 17a can be connected with a substituate source through a, preferably automatic, substituate port 18. By means of the substituate pump 17, substituate may be introduced via pre-dilution or via post-dilution through associated lines 13 or 14 into line sections, e.g. into the arterial section 9 or into a venous section 23a (between a blood chamber 19a of a blood filter 19 and a venous air separation chamber 21), of the blood circuit 1.

The blood filter 19 comprises the blood chamber 19a which is connected to the arterial section 9 and to the venous section 23. A dialysate chamber 19b of the blood filter is connected to a dialysis liquid inlet line 31a leading to the dialysate chamber 19b and to a dialysate outlet line 31b leading away from the dialysate chamber 19b.

The dialysis liquid inlet line 31a optionally comprises a valve V24 by which the flow within the dialysis liquid inlet line 31a may be interrupted. The dialysate outlet line 31b optionally comprises a valve V25 by which the flow within the dialysate outlet line 31b may be interrupted.

The dialysis liquid inlet line 31a is further optionally connected to a compressed air source 26 by means of another internal valve of the apparatus. The compressed air source 26 may be provided as a part or component of the treatment apparatus 4 or as a part separated thereof. A pressure sensor 37 may be provided downstream of the compressed air source 26.

The fluid connection 34 indicated in FIG. 1 by a dash-dotted line represents a connection between the arterial section 9 and the venous section, here upstream of the addition site 14 for substituate (postdilution). The fluid connection 34 may, amongst others, serve the simple automatic draining of the arterial section 9 or the inlet line 8 of the extracorporeal blood circuit. The fluid connection 34 may in addition ensure that the pressure which is built up according to the present disclosure cannot unintentionally decrease via the arterial section 9.

The fluid connection 34 may be achieved by a factory-made connection section 24 with e.g. the venous section 23 or 23a. The connection section 24 may allow a fluid connection with the venous section 23 or 23a.

The fluid connection 34 may be easily achieved by disconnecting the arterial section 9 from the patient and connecting it to e.g. the venous section 23 or 23a, for example using the optional connection section 24.

To maintain the pressure, one or several different measures may be used instead of (or in addition to) the fluid connection 34. Thus, a decrease of the pressure through the arterial side of the blood circuit may be prevented for example by an occluding pump, here the blood pump 11, or by closing the arterial patient hose clamp 6.

In order to effect the method for emptying the extracorporeal blood circuit 1 from blood after the completion of the treatment, in particular to empty the blood chamber 19a of the blood filter 19, the following steps a) to f) may be executed once or several times by the control and/or closed-loop control device 29 according to the present disclosure. In this, it is assumed that it has been ensured, preferably automatically, that the venous air separation chamber 21 is only partially, i.e. not completely, filled with blood.
a) interrupting a fluid connection between the venous air separation chamber 21 on the one hand and the single-needle chamber 36 (as an example for the atmosphere or the exterior) on the other hand by closing a valve, here e.g. the valve 35;
b) closing the venous patient hose clamp 7 or preventing the flow along the venous blood return line 23 downstream of the blood filter 19 in a different way;
c) introducing substituate or dialysis liquid into the blood circuit 1, in particular into the arterial blood withdrawal line 9 or into the venous blood return line 23;
d) measuring the pressure prevailing in the arterial blood withdrawal line 9 and/or in the venous blood return line 23 using the pressure sensor (33a, 33b);
e) establishing a connection between the venous air separation chamber 21 and the single-needle chamber 36 of the extracorporeal blood circuit 1 by opening the valve 35, when the measured pressure reaches or exceeds a predetermined first value; and
f) interrupting a connection between the venous air separation chamber 21 and the single-needle chamber 36 of the extracorporeal blood circuit 1 by closing the valve 35 and opening the patient hose clamp 7, in both cases after the measured pressure has decreased to a predetermined second value.

In the present embodiment (double-needle method) the single-needle chamber 36 serves as a ventilation device for the venous air separation chamber 21 and represents therefore the atmosphere or the exterior.

Alternatively, it is conceivable that a valve other than the valve 35 represents a connection between the atmosphere/the exterior and the interior of the extracorporeal blood circuit 1 and thus said connection between the atmosphere/the exterior and the interior is established or interrupted by opening or closing said other valve.

Alternatively or additionally, the substituate may be introduced by the blood pump 11 and not by the substituate pump 17. For this purpose, the arterial patient hose clamp 6 is closed and substituate is introduced from a reservoir for substituate into the extracorporeal blood circuit 1 through an inlet line 8.

The control and/or the closed-loop control device 29 may be part of a control and/or closed-loop control device of the treatment apparatus 4.

The arrangement of FIG. 1 encompasses an optional detector 15 for detecting air and/or blood. The arrangement of FIG. 1 further encompasses one or two pressure sensors 33a, 33b at the illustrated points in FIG. 1.

In FIG. 1, the single-needle chamber 36 is used as buffer and/or compensation tank, in particular during or after a double-needle method by which the patient is connected to the extracorporeal blood circuit 1 via two blood lines 9, 23. It is obvious for the person skilled in the art that any other compensation tank may be taken into consideration as well.

The present disclosure is not limited to the embodiment as described above, this is considered only for illustrative purposes. Also a time regulation as described supra may be used instead of or in addition to pressure regulation as described with respect to FIG. 1. Furthermore, the disclosure is not limited to emptying the content or parts thereof while a connection with the vascular system still exists.

### LIST OF REFERENCE NUMERALS

1 extracorporeal blood circuit
2 blood cassette
4 treatment apparatus
5 access device, e.g. arterial connection needle
6 arterial patient hose clamp
7 venous patient hose clamp
8 supply line
9 arterial section or arterial blood withdrawal line or arterial patient line
11 blood pump
13 addition site for substituate (predilution)
14 addition site for substituate (postdilution)
15 arterial air/blood detector
17 second conveying device, e.g. a substituate pump
17a substituate line
18 automatic substituate port
19 blood filter
19a blood chamber
19b dialysate chamber
21 venous air separation chamber
23 venous section or venous blood return line
23a venous section
24 connection site, connection section
25 venous substituate/blood detector
26 compressed air source
27 access device, e.g. venous connection needle
29 control or closed-loop control device
31a dialysis liquid inlet line
31b dialysate outlet line
33a, b pressure sensors
34 fluid connection
35 single-needle valve
36 single needle chamber
37 pressure sensor
V24 valve
V25 valve

1. A control device configured for controlling, in interaction with a blood treatment apparatus, a method for removing blood from a blood filter or extracorporeal blood circuit used for a blood treatment of a patient after completion of the blood treatment,
wherein the extracorporeal blood circuit comprises:
the blood filter having a blood chamber and a dialysis chamber between which a membrane is arranged;
an arterial blood line or blood withdrawal line connected to the blood chamber;
a venous blood line or blood return line connected to the blood chamber; and
a valve for selecting between establishing or interrupting a fluid connection between a line interior of the extracorporeal blood circuit and atmosphere at an exterior of the extracorporeal blood circuit;
wherein the blood treatment apparatus comprises:
at least one blood pump arranged along the extracorporeal blood circuit for conveying blood within the line interior of the extracorporeal blood circuit:
at least one conveying device for introducing substituate or dialysis liquid into the arterial blood line or blood withdrawal line or into the venous blood line or blood return line;
at least one venous patient hose clamp for interrupting a flow through a section of the venous blood line or blood withdrawal line;
at least one actuation device for actuating the valve; and
at least one pressure sensor for measuring a pressure prevailing in the arterial blood line or blood withdrawal line or in the venous blood line or blood return line;
wherein the method comprises:
a) interrupting a connection between the atmosphere and the line interior of the extracorporeal blood circuit by closing the valve;
b) closing the at least one venous patient hose clamp;
c) introducing substituate or dialysis liquid into: (i) the arterial blood line or the blood withdrawal line, or (ii) the venous blood line or the blood return line, and/or establishing an increased pressure, via a pump used for the introducing, in (i) the arterial blood line or the blood withdrawal line, or (ii) the venous blood line or the blood return line;
d) establishing the fluid connection between the atmosphere/the exterior and the line interior of the extracorporeal blood circuit by opening the valve either: (i) in response to the measured pressure reaching or exceeding a predetermined first value or (ii) when a predetermined first time period has elapsed since beginning the introducing substituate or dialysis liquid since the beginning of the establishing or increasing pressure in (i) the arterial blood line or the blood withdrawal line, or (ii) the venous blood line or the blood return line with the closed at least one venous patient hose clamp; and
f) interrupting the fluid connection between the atmosphere and the line interior of the extracorporeal blood circuit by closing the valve and opening the at least one venous patient hose clamp either (i) in response to the measured pressure decreasing to a predetermined second value or (ii) when a predetermined second time period has elapsed since the valve was opened in step d).

2. The control device according to claim 1, wherein the predetermined first value for the pressure is between 1 and 2 bar or the predetermined first time period is between 2 sec and 5 sec.

3. The control device according to claim 1, wherein the predetermined second value is between 0 and 0.5 bar or the predetermined second time period is between 1 sec and 3 sec.

4. The control device according to claim 1, wherein when executing the method, a free end of the arterial blood line or blood withdrawal line is connected in fluid communication to a further section of the venous blood line or blood return line.

5. The control device according to claim 1, wherein the extracorporeal blood circuit comprises a venous air separation chamber; and
wherein the method begins only after it has been ascertained that the venous air separation chamber is not completely filled with liquid, or it is not filled with liquid beyond a predetermined level.

6. The closed control device according to claim 1, wherein the extracorporeal blood circuit comprises a venous air separation chamber and a single-needle chamber, and wherein the valve is arranged between the single-needle chamber and the venous air separation chamber.

7. The control device according to claim 1, wherein the steps a) to e) of the method for removing blood are performed several times in succession.

8. A medical treatment apparatus comprising:
at least one extracorporeal blood circuit with a line interior and comprising an arterial blood line or blood withdrawal line;
at least one blood pump arranged along the extracorporeal blood circuit for conveying blood within the line interior of the extracorporeal blood circuit;
at least one conveying device for introducing substituate or dialysis liquid into the arterial blood line or blood withdrawal line; and
at least one control device according to claim 1.

9. The medical treatment apparatus according to claim 8, wherein the extracorporeal blood circuit is, at least in section thereof, part of a blood cassette.

10. A digital storage medium in the form of a disk, CD, or DVD or EPROM, with electronically readable control signals, configured for configuring the control device according to claim 1.

11. A computer product with a program code saved on a machine-readable carrier for configuring the control device according to claim 1.

12. A computer program with a program code for configuring the control device according to claim 1.

13. The control device according to claim 1, wherein the method further comprises measuring, using the pressure sensor, the pressure prevailing in: (i) the arterial blood line or blood withdrawal line or (ii) the venous blood line or blood return line.

14. The control device according to claim 1, wherein the method further comprises measuring, using the pressure sensor, the pressure prevailing in: (i) the arterial blood line or blood withdrawal line and (ii) the venous blood line or blood return line.
